(19) United States
(12) Patent Application Publication
(10) Pub. No.: US 2015/0118257 A1
(43) Pub. Date: Apr. 30, 2015

(54) Methods and Compositions for Manipulating the Immune System

(71) Applicant: The Wistar Institute of Anatomy and Biology, Philadelphia, PA (US)

(72) Inventors: Hui Hu, Vestavia Hills, AL (US); Haikun Wang, Philadelphia, PA (US)

(73) Assignee: The Wistar Institute of Anatomy and Biology

(21) Appl. No.: 14/395,762

(22) PCT Filed: Mar. 15, 2013
(86) PCT No.: PCT/US13/32027

§ 371 Date: Oct. 20, 2014

Related U.S. Application Data
(60) Provisional application No. 61/636,425, filed on Apr. 20, 2012.
(60) Provisional application No. 61/637,136, filed on Apr. 23, 2012.

(51) Int. Cl. C12N 15/113

(52) U.S. Cl. CPC C12N 15/113; C12N 2310/17
USPC 424/184.1

(57) ABSTRACT

Methods and compositions, e.g., therapeutic agents, are provided for modulating gene and/or protein expression of Forkhead Box protein 1 (Foxp1), particularly Foxp1A and Foxp1D, in CD4+ T cells. Such modulation permits manipulation of the B cell response and antibody production and activity, without depleting the number, production or activity of the T cells. In one aspect, methods and compositions for increasing or up regulating the nucleic acid and/or protein expression of Foxp1A, Foxp1D or a combination thereof in the subject's cells in vivo, inhibits or suppresses B cell response and antibody production or activity thereof in the subject. This aspect is useful for treating diseases characterized by excessive B cell response or antibody production or activity, such as autoimmune disorders

### STATEMENT REGARDING FEDERALLY SPONSORED RESEARCH OR DEVELOPMENT

This invention was made with government support under Grant Nos. 1R56A1088102 awarded by the National Institutes of Health. The government has certain rights in this invention.

### INCORPORATION-BY REFERENCE OF MATERIAL SUBMITTED IN ELECTRONIC FORM

Applicant hereby incorporates by reference the Sequence Listing material filed in electronic form herewith. This file is labeled WST135PCT_ST25.txt”, was created on 14 Mar. 2013, and is 41.8 KB in size.

### BACKGROUND OF THE INVENTION

Aberrant activity of the humoral immune system, e.g., B cell activation and production of antibodies, can result in a variety of disorders. Specifically, excessive antibody activity can result in inflammation, allergic reactions or anaphylaxis, and autoimmune disorders. Conversely, poor antibody response often results in increased susceptibility to infection, cancer or other diseases. However, manipulation of the antibody response is not a simple process, because it is intimately linked with the production and activity of the cellular immune system. Attempts to manipulation cellular immunity thus can impact antibody production or activity, which is necessary for health.

There remains a need in the art for effective mechanisms for the successful modulation of both arms of the immune system to permit treatment of a variety of disorders.

### SUMMARY OF THE INVENTION

In one aspect, a method of modulating the immune response in a mammalian subject comprises modulating the expression or activity of Foxp1, or an isoform thereof, or a combination thereof in the cells of the subject. The Foxp1 may be the full-length isoform, Foxp1 SEQ ID NO: 1 and/or the shorter isoform Foxp1D SEQ ID NO: 2. Preferably, this modulation occurs in the T cells, e.g., CD4+ cells or a subset thereof, i.e., T follicular helper cells.

In one aspect, this method involves increasing or up regulating the nucleic acid and/or protein expression of Foxp1A, Foxp1D or a combination thereof in the subject's cells in vivo, thereby inhibiting or suppressing B cell response and antibody production or activity in the subject. The B cell response and antibody production or activity is reduced or inhibited without depleting the T cell population or activity.

In another aspect, this method involves decreasing or down regulating the nucleic acid or protein expression of Foxp1A, Foxp1D or a combination thereof in the subject's T cells in vivo, thereby enhancing B cell response and antibody production or activity in the subject. The B cell response and antibody production or activity is enhanced without depleting the T cell population or activity.

In another aspect, a method of treating a mammalian subject having a disease characterized by excessive B cell response and antibody production or activity comprises administering to a subject in need thereof a therapeutic reagent that up-regulates the expression of Foxp1A, Foxp1D or a combination thereof in T cells of the subject.

In another aspect, a method of treating a mammalian subject having a disease characterized by insufficient B cell response and antibody production or activity comprises administering to a subject in need thereof a therapeutic reagent that down-regulates the expression of Foxp1A, Foxp1D or a combination thereof in T cells of the subject.

In still other aspect, therapeutic or prophylactic compositions for modulating the expression of Foxp1A, Foxp1D, or a combination thereof, and a pharmaceutically acceptable carrier or diluent are provided.

Other aspects and advantages of these compositions and methods are described further in the following detailed description of the preferred embodiments thereof.

### BRIEF DESCRIPTION OF THE DRAWINGS

FIG. 1 is a picture of a Western gel of murine Foxp1 expression in näive and activated murine CD4⁺ T cells in which β-actin was used as loading control. See Example 1. The gel shows that Foxp1D is induced in activated T cells by T cell receptor (TCR) stimulation.

FIG. 2 is a diagram of the generation of the transgenes used to create Foxp1A and Foxp1D conditional transgenic mice described in Example 2.

FIGS. 3A and 3B are a series of histograms produced after infecting Foxp1A^{Tg}Cd4^{Cre} and wild-type (Control, Ctrl) mice with PR8 flu viruses as described in Example 3. FIG. 3A shows the results of CXCR5⁺PD-1⁺ Tfh cell staining on CD44^{hi}CD62L^{lo}CD4⁺ T cells on Day 10 post-infection: i.e., infected control, 33%, and infected FOXP1A^{Tg}Cd4^{Cre} cells, 16%. FIG. 3B shows the results of gating of the germinal center (GC, PNA⁺FAS⁺) B cells on B220⁺IgD^{low} cells on Day 10: uninfected Foxp1A^{Tg}Cd4^{Cre} cells, 1.2%, infected controls (Ctrl), 16% and infected Foxp1A^{Tg}Cd4^{Cre} cells, 2.3%; and on Day 37, infected controls, 5% and infected Foxp1A^{Tg}Cd4^{Cre}, 0.8%.

FIGS. 4A and 4B are a series of histograms produced after infecting Foxp1D^{Tg}Cd4^{Cre} and wild-type (Control, Ctrl) mice with PR8 flu viruses and analyzing them as described in FIG. 3 and Example 3 below. FIG. 4A shows the results of CXCR5⁺PD-1⁺ Tfh cell staining gated on CD44^{hi}CD62L^{lo}CD4⁺ T cells on Day 10 shown in both the infected control, 21%, and infected Foxp1D^{Tg}Cd4^{Cre} cells, 4%. FIG. 4B shows the results of the germinal center (GC, PNA⁺FAS⁺) B cells gated on B220⁺IgD^{low} cells on Day 10 for uninfected Foxp1D^{Tg}Cd4^{Cre} cells, 0.5%, for infected Ctrl cells, 12% and for infected Foxp1D^{Tg}Cd4^{Cre} cells, 0.6%; and on Day 37 for infected Ctrl cells, 5% and for infected Foxp1D^{Tg}Cd4^{Cre} cells, 0.3%.

FIG. 5A is a flow chart diagram of the adoptive transfer experiment of Example 4. Naïve, purified CD4⁺ T cells obtained from wild-type OT-II transgenic (Ctrl) mice or OT-II^{Tg}Foxp1^{f/f}Cre-ERT2⁺Rosa^{YFP} (all Foxp1 deleted) mice, were treated with tamoxifen for two days in vitro. These cells were sorted with wild-type (Ctrl) or YFP⁺ cells and transferred (or as a mixed co-transfer) into Ly5.1⁺SMARTA TCR transgenic mice or intact Ly5.1⁺C57BL/6 recipient mice. The recipient mice were immunized with NP-OVA.

FIG. 5B is a series of 4 histograms generated 5 days after immunization described in FIG. 5A. The splenic cells (Spl) and draining lymph nodes (mLN) of the recipient mice were analyzed for CXCR5⁺PD-1⁺ Tfh staining gated on CD44^{hi}CD62L^{lo}CD4⁺ T cells.

FIG. 5C are two histograms generated from mixed co-transfer experiments of Example 4. mLN of the recipient mice were analyzed on Day 5 post immunization for CXCR5⁺PD-1⁺ Tfh staining gated on CD44^{hi}CD62L^{lo}CD4⁺ T cells. These data show that Foxp1 deletion leads to dramatically enhanced Tfh responses.

### DETAILED DESCRIPTION OF THE INVENTION

This invention provides compositions, e.g., therapeutic agents, and methods that modulate gene and protein expression of Forkhead Box protein 1 (Foxp1) expression, particularly Foxp1A and Foxp1D. The inventors have determined that modulation of the expression of the transcription factor Foxp1 in T cells, particularly in T helper cells, permits the manipulation of the humoral immune system. The compositions and methods described herein are based on the inventors' finding that the Foxp1 pathway has a novel negative regulation of T helper cell, i.e., CD4+ T follicular helper cells (Tfh) development by mechanisms including a negative feedback loop of Foxp1D.

In newly generated Foxp1D isoform-specific conditional transgenic mice, the inventors found that Foxp1D transgene inhibits TCR signaling and T cell activation, and dramatically inhibits Tfh development and the subsequent germinal center formation and B cell response to antigen challenge. Such results are verified by the complementary experiments in which FOXP1-deficient T cells are used. See Examples 1-3 below. Further in vivo studies (Example 4) demonstrate that the preferential development of Tfh cells in the absence of Foxp1 occurs at an early stage. A robust germinal center response was induced, indicating that the downregulation of Foxp1 stimulated a more robust B cell response.

Thus the methods and therapeutic agents discussed herein modulate gene and protein expression of Forkhead Box protein 1 (Foxp1) expression, particularly Foxp1A, Foxp1D or combinations of both transcription factors. The compounds and methods of the present invention have applications in therapy of diseases mediated by excessive humoral (B cell/antibody) response, development and/or activity or insufficient humoral response, development and/or activity, either alone or in combination with other therapies.

### I. Definitions and Components of the Invention

Technical and scientific terms used herein have the same meaning as commonly understood by one of ordinary skill in the art to which this invention belongs and by reference to published texts, which provide one skilled in the art with a general guide to many of the terms used in the present application. The following definitions are provided for clarity only and are not intended to limit the claimed invention.

The forkhead box (Fox) proteins constitute a large transcription factor family with diverse functions in development, cancer and aging. Transcription factor Foxp1 is expressed in many tissues and is a critical transcriptional regulator in B lymphopoiesis (Hu et al, 2006 Nat. Immunol. 7, 819-826). In T cells, Foxp1 has been shown to be an essential regulator in the generation of quiescent naive T cells during thymocyte development (Feng et al, 2010 Blood, 115:510-518). In the periphery, Foxp1 pathway has been shown to be a quiescence pathway that restrains T cell activation (Feng et al, 2011 Nat. Immunol. 12, 544-550). Now, new evidence shows that Foxp1 plays a critical role in the development of T follicular helper cells (see Examples below).

NCBI Gene ID No. 27086 provides the human gene information for the Foxp1 gene of homo sapiens. The DNA sequence for one transcript variant of the 7201 bp human Foxp1 mRNA sequence is reported at NBCI Reference Sequence NM_{—}032682.5 (SEQ ID NO. 1). This full length isoform Foxp1A has a protein coding region spanning nt 527 through nt 2560 of SEQ ID NO. 1, encoding a 677 amino acid protein (SEQ ID NO: 2). Another isoform is Foxp1D (also known as Foxp1 isoform 6 (NCBI Reference Sequence NM_{—}001244813.1 for the nucleic acid sequence and NP 0012317342.1 for the protein sequence; SEQ ID NOs. 3 and 4, respectively). Other variants are known and can be obtained commercially from e.g., GeneCopoeia, among other commercial sources. Similarly one may obtain murine nucleotide and protein sequences of Foxp1 from similar sources (see e.g., NCBI Ref Nos. NM_{—}001197322.1, NM_{—}053202.1 and BC064764.1). In mice, Foxp1 has four isoforms, as described in Wang et al, July 2003, J. Biol. Chem., 278(27):24259-24268. The full-length Foxp1A and a shorter Foxp1D which is missing the 5′ 37-polygluamine sequence of the full-length sequence. The full-length Foxp1A and a shorter Foxp1D which is missing the 5′ 37-polygluamine sequence of the full-length sequence are the two major isoforms that were found to be expressed in T lineage cells. Homologous sequences are found in humans and other mammals. All such published sequences for Foxp1 variants are incorporated herein by reference.

In one embodiment, the compositions and methods described herein target Foxp1A as set forth in SEQ ID NO: 1. Thus in some embodiments, the term “Foxp1” refers to any Foxp1 protein, peptide, or polypeptide or isoform, including naturally occurring or deliberated mutated or genetically engineered sequences, having Foxp1 family activity such as encoded by SEQ ID NO: 1. In other embodiments, the Foxp1 isoform used is Foxp1D (SEQ ID NO: 3). In other embodiments, the term Foxp1 includes any nucleic acid sequence encoding a Foxp1 protein, peptide, or polypeptide of mammalian origin, including naturally occurring or deliberated mutated or genetically engineered sequences. In still other embodiments, Foxp1-related molecules include polymorphisms or single nucleotide polymorphisms of Foxp1, Foxp1 homologs, and Foxp1 splice and transcript variants. Other human isoforms of Foxp1, isoforms 1-8 are identified under the NCBI Gene ID No. 27086. Throughout the following application the terms “Foxp1A”, “Foxp1D” or “Foxp1A/Foxp1D” can be used interchangeably to refer to full length Foxp1A or one of its fragments or shorter isoforms, such as Foxp1D.

The term “target nucleic acid” as used herein means any nucleic acid sequence of Foxp1, but preferably Foxp1A, Foxp1D or a combination thereof, whose expression or activity is to be modulated. The target nucleic acid can be DNA or RNA.

The term “target cells” as used herein refers to those cells in which Foxp1, preferably Foxp1A and Foxp1D, or a combination of same are to suppressed or overexpressed. In one embodiment, the target cell is a helper T cell, e.g., CD4+ T cell. In another embodiment the target cells are T follicular helper cells (Tfh) cells. Other target cells will be obvious from the description below.

The term “homolog” or “homologous” as used herein with respect to any target sequence (e.g., Foxp1A, etc.) means a nucleic acid sequence or amino acid sequence having at least 35% identity with the mRNA or protein sequence, respectively, of the target sequence, e.g., of a specific Foxp1A isoform, used for comparison and encoding a gene or protein having substantially similar function to that of the reference sequence. Such homologous sequences can be orthologs, e.g., genes in different species derived from a common ancestor. In other embodiments, the homolog can have at least 40, 50, 60%, 70%, 80%, 90% or at least 99% identity with the respective human target sequence. In one embodiment, the homolog is that of a non-human mammalian species, e.g., such as the murine Foxp1A and Foxp1D identified in the examples below. Based on the known and publically available sequences of these transcription factors and the available computer programs readily available, such as the BLAST program, one of skill in the art can readily obtain full-length homologs, orthologs or suitable fragments of the target genes or proteins referred to herein from a mammalian species.

The term “hairpin” and “stem-loop” can be used interchangeably and refer to stem-loop structures. The stem results from two sequences of nucleic acid or modified nucleic acid annealing together to generate a duplex. The loop lies between the two strands comprising the stem. The term “loop” refers to the part of the stem-loop between the two homologous regions (the stem) that can loop around to allow base-pairing of the two homologous regions. The loop can be composed of nucleic acid (e.g., DNA or RNA) or non-nucleic acid material(s), referred to herein as nucleotide or non-nucleotide loops. A non-nucleotide loop can also be situated at the end of a nucleotide molecule with or without a stem structure.

The term “complementary” and “complementarity” are interchangeable and refer to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G). Complete or 100% complementarity refers to the situation in which each nucleotide unit of one polynucleotide strand or region can hydrogen bond with each nucleotide unit of a second polynucleotide strand or region. Complementarities less than 100%, e.g., 95%, 90%, 85%, refers to the situation in which 5%, 10% or 15% of the nucleotide bases of two strands or two regions of a stated number of nucleotides, can hydrogen bond with each other.

The term “gene” as used herein means a nucleic acid that encodes a RNA sequence including but not limited to structural genes encoding a polypeptide.

The term “sense region” as used herein means a nucleotide sequence of a small nucleic acid molecule having complementary to a target nucleic acid sequence. In addition, the sense region of a small nucleic acid molecule can comprise a nucleic acid sequence having homology with a target nucleic acid sequence.

The term “antisense region” as used herein means a nucleotide sequence of a small nucleic acid molecule having a complementarity to a target nucleic acid sequence. It can also comprise a nucleic acid sequence having complementarity to a sense region of the small nucleic acid molecule.

The term “modulate” or “modulates” means that the expression of the gene or level of RNA molecule or equivalent RNA molecules encoding one or more protein or protein subunits or peptides, or the activity of one or more protein subunits or peptides is up regulated or down regulated such that the expression, level, or activity is greater than or less than that observed in the absence of the modulator. The term “modulate” includes “inhibit” or over-express, depending upon the use.

The phrase “disease mediated by a dysfunctional humoral immune system” can be a disease caused or negatively impacted by excessive B cell (antibody) production or activity, such as an autoimmune disease, allergy or anaphylaxis, or a disease caused or negatively impacted by insufficient B cell (antibody) production or activity, such as infection.

As used herein, the term “subject”, “patient”, or “mammalian subject” includes primarily humans, but can also be extended to include domestic animals, such as dogs and cats, and certain valuable animals, such as horses, farm animals, laboratory animals (e.g., mice, rats) and the like.

The term “B cell” refers to a lymphocyte that matures into a plasma cell that produces an antibody, or memory B cell which can mature into a plasma cell that produces an antibody after reencountering the same antigen.

As used herein, the term “antibody,” refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), diabodies, Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

### II. Compositions Useful in the Invention

As disclosed herein, the compositions described herein modulate the expression of, or target, Foxp1, preferably Foxp1A and/or Foxp1D, in target mammalian T helper cells or Tfh cells. A therapeutic or prophylactic composition comprises a nucleic acid construct that modulates the expression of Foxp1A, Foxp1D, or a combination thereof, and a pharmaceutically acceptable carrier or diluent, such as saline or buffered saline.

In one embodiment, the compositions described herein can be used to increase or up regulate the expression of Foxp1A, Foxp1D or a combination thereof in the subject's cells in vivo, thereby inhibiting or suppressing B cell response and/or antibody production and/or activity in the subject. For example, in one embodiment the composition comprises a nucleic acid construct comprising a sequence encoding Foxp1A, Foxp1D or a combination thereof under the regulatory control of a promoter that overexpresses or can overexpress the Foxp1A or Foxp1D sequence in the target cells. For example, the nucleic acid construct can include a viral vector or plasmid vector containing which has one or more iterations of the Foxp1A and/or Foxp1D sequence under the control of a strong constitutive or inducible promoter so that the expression of the Foxp1A and/or Foxp1D RNA is overexpressed in the target T cells.

In another embodiment, the compositions described herein can be used to decrease or down regulate the expression of Foxp1A and/or Foxp1D or a combination thereof in the subject's cells in vivo, thereby enhancing B cell response and/or antibody production and/or activity in the subject. For example, in one embodiment the composition comprises a nucleic acid construct comprising a sequence that reduces or suppresses the expression of Foxp1A, Foxp1D or a combination thereof in the target cells. For example, the down regulating composition can include a nucleic acid construct comprising a short nucleic acid molecule selected from the group consisting of a short hairpin RNA (shRNA), a short interfering RNA (siRNA), a double stranded RNA (dsRNA), a micro RNA, and an interfering DNA (DNAi) molecule, optionally under the control of a suitable regulatory sequence.

Therefore compositions useful herein can employ a variety of components and be achieved in multiple ways.

A. Short Nucleic Acid Molecules

A short nucleic acid molecule useful in the compositions and in the methods described herein is any nucleic acid molecule capable of inhibiting or down-regulating Foxp1 gene expression. Typically, short interfering nucleic acid molecules are composed primarily of RNA, and include siRNA or shRNA, as defined below. A short nucleic acid molecule may, however, include nucleotides other than RNA, such as in DNAi (interfering DNA), or other modified bases. Thus, the term “RNA” as used herein means a molecule comprising at least one ribonucleotide residue and includes double stranded RNA, single stranded RNA, isolated RNA, partially purified, pure or synthetic RNA, recombinantly produced RNA, as well as altered RNA such as analogs or analogs of naturally occurring RNA. In one embodiment the short nucleic acid molecules of the present invention is also a short interfering nucleic acid (siNA), a short interfering RNA (siRNA), a double stranded RNA (dsRNA), a micro RNA (μRNA), and/or a short hairpin RNA (shRNA) molecule. The short nucleic acid molecules can be unmodified or modified chemically. Nucleotides of the present invention can be chemically synthesized, expressed from a vector, or enzymatically synthesized.

In some embodiments, the short nucleic acid comprises between 18 to 60 nucleotides. In another embodiment, the short nucleic acid molecule is a sequence of nucleotides between 25 and 50 nucleotides in length. In still other embodiments, the short nucleic acid molecule ranges up to 35 nucleotides, up to 45, up to 55 nucleotides in length, depending upon its structure. These sequences are designed for better stability and efficacy in knockdown (i.e., reduction) of Foxp1 gene expression. In one embodiment, the nucleic acid molecules described herein comprises 19-30 nucleotides complementary to a Foxp1 nucleic acid sense sequence, particularly an open reading frame of Foxp1. In one embodiment, the nucleic acid molecules described herein comprises 19-30 nucleotides complementary to a Foxp1 antisense nucleic acid sequence strand. In one embodiment, the nucleic acid molecules described herein comprises 19-30 nucleotides complementary to a Foxp1 nucleic acid sense sequence and comprises 19-30 nucleotides complementary to a Foxp1 antisense nucleic acid sequence strand.

1. siRNA Molecules

In one embodiment, a useful therapeutic agent is a small interfering RNA (siRNA) or a siRNA nanoparticle. siRNAs are double stranded, typically 21-23 nucleotide small synthetic RNA that mediate sequence-specific gene silencing, i.e., RNA interference (RNAi) without evoking a damaging interferon response. siRNA molecules typically have a duplex region that is between 18 and 30 base pairs in length. Foxp1 siRNAs are designed to be homologous to the coding regions of Foxp1 mRNA (e.g., SEQ ID NO: 1) and suppress gene expression by mRNA degradation. The siRNA associates with a multi protein complex called the RNA-induced silencing complex (RISC), during which the “passenger” sense strand is enzymatically cleaved. The antisense “guide” strand contained in the activated RISC then guides the RISC to the corresponding mRNA because of sequence homology and the same nuclease cuts the target mRNA, resulting in specific gene silencing. The design of si/shRNA preferably avoids seed matches in the 3′UTR of cellular genes to ensure proper strand selection by RISC by engineering the termini with distinct thermodynamic stability. A single siRNA molecule gets reused for the cleavage of many target mRNA molecules. RNAi can be induced by the introduction of synthetic siRNA.

In one embodiment, a siRNA molecule of the invention comprises a double stranded RNA wherein one strand of the RNA is complimentary to the RNA of Foxp1. In another embodiment, a siRNA molecule of the invention comprises a double stranded RNA wherein one strand of the RNA comprises a portion of a sequence of RNA having Foxp1 sequence. SEQ ID Nos: 5 and 6 illustrate two exemplary siRNAs for Foxp1. Synthetic siRNA effects are short lived (a few days) probably because of siRNA dilution with cell division and also degradation.

In one embodiment, siRNA without any chemical modification having high stability and specificity for Foxp1, are useful as therapeutics alone, or in combination with other therapies for cancer. In another embodiment, siRNA oligonucleotides targeting Foxp1 are complexed or conjugated to a polymer or any other material that stabilizes siRNA, for use as therapeutics alone, or in combination with other therapies for cancer.

Among such stabilizing polymers and materials are polyethyleneimine (PEI), which may be conjugated to siRNA, resulting in the generation of nanocomplexes of about 50 nm, as described in Cubillos-Ruiz J R, et al, 2009 J. Clin. Invest., 119(8):2231-44, incorporated by reference herein. In another embodiment, such a stabilizing material is chitosan. In one embodiment, the siRNA is in a stable composition, with or without conjugation, with cholesterol. In still other embodiments, siRNA may be combined with conjugates such as a lipid, a cationic lipid, a phospholipid, and a liposome.

In another embodiment, the siRNA is in a stable composition, with or without conjugation, to an antibody or fragment thereof that permits the siRNA to be preferentially targeted. In one embodiment, the antibody is an antibody or fragment to a desirable molecule, such as an IL7 receptor. In another embodiment, the antibody is an antibody or fragment to a T cell surface marker, a T cell receptor or a chimeric receptor which also permits targeting. For example, in one another embodiment, the siRNA are linked to thiolated F(ab)2 fragments of monoclonal antibodies targeting T cell surface markers (e.g., CD3, CTLA4, CD44, CD69 or CD25). In another embodiment, the antibody or fragment is to a T cell receptor or chimeric receptor. T cell receptors or chimeric receptors for association with, or co-expression with the siRNA include without limitation, TCRs against human antigens. Among such useful TCRs include those that have been transduced in adoptively transferred T cells (reviewed in Trends Biotechnol. 2011 November; 29(11):550-7). In one embodiment, the TCR is the receptor that binds human carcinoembryonic antigen (Parkhurst M R et al, 2011 Mol. Ther., 19(3):620-6), NY-ESO-1 (Robbins P F et al, 2011 J. Clin. Oncol., 29(7):917-24), MAGE-A3 (Chinnasamy N et al 2011 J. Immunol., 186(2):685-96) and MART-1, gp100 and p53 (Morgan R A et al, 2006 Science, 314(5796):126-9). Association with such TCRs is described in Westwood et al, 2005, cited herein. Examples of chimeric receptors useful in the compositions and methods described herein are chimeric receptors against the antigens CD19 (Kolos M, et al, 2011 Sci Transl. Med., 3(95):95ra73) and Epstein Barr virus (Fondell, J D et al, 1990 J. Immunol., 144(3):1094-103). Other chimeric receptors have also targeted mesothelin (Moon E K et al, 2011 Clin Cancer Res., 17(14):4719-30) and the folate receptor (Song D G et al, 2011 Cancer Res., 71(13):4617-27).

2. shRNA Molecules

In another embodiment, the short nucleic acid molecule is a small hairpin RNA (shRNA). A shRNA molecule useful in the methods and compositions described herein is generally defined as an oligonucleotide containing the about 18-23 nucleotide siRNA sequence followed by a ˜9-15 nt loop and a reverse complement of the siRNA sequence. The loop nucleotides generally form a non-coding sequence. Examples of commercially available shRNA sequences targeting human Foxp1 can be readily generated by one of skill in the art.

shRNAs can be cloned in plasmids or in non-replicating recombinant viral vectors to endogenously/intracellularly express shRNA, which is subsequently processed in the cytoplasm to siRNA. The shRNA effects are longer lasting because they are continually produced within the cells and thus have an effect that lasts the duration of the cell's life.

B. Recombinant Vectors Carrying a FOXP1A and/or FOXP1D RNA Expressing Construct or a FOXP1A and/or FOXP1D siRNA or shRNA Inhibiting Construct

These Foxp1A/Foxp1D sequences can be produced in plasmid based systems or viral vector systems, of which many are commercially available. Suitable plasmid and viral vectors are well known to those of skill in the art and are not a limitation of the present invention. Briefly, the nucleic acid sequence encoding the Foxp1A/Foxp1D sequences is inserted into a vector or plasmid which contains other optional flanking sequences, a promoter, an mRNA leader sequence, an initiation site and other regulatory sequences capable of directing the multiplication and expression of that sequence in vivo or in vitro. As used herein, a vector may include any genetic element including, without limitation, naked DNA, a phage, transposon, cosmid, episome, plasmid, bacteria, or a virus. As used herein, the term vector refers to a genetic element which expresses, or causes to be expressed, the desired construct that overexpresses the Foxp1A/Foxp1D factors or inhibits the expression of Foxp1A/Foxp1D in the target cell ex vivo or in vivo.

As well known in the art, a nucleotide sequence (which encodes the Foxp1A/Foxp1D encoding sequences or inhibitory sequences) is inserted into an expression vector, transformed or transfected into an appropriate host cell and optionally cultivated under conditions suitable for expression. These procedures are generally known in the art, as described generally in Sambrook et al., MOLECULAR CLONING, A LABORATORY MANUAL, Cold Spring Harbor Press, Cold Spring Harbor, N.Y. (1989).

However, because they are easy to deliver, non-replicating recombinant viral vectors are commonly used for RNA or shRNA expression. Thus, in one embodiment, the vector is a non-pathogenic virus. In another embodiment, the vector is a non-replicating virus. In one embodiment, a desirable viral vector may be a retroviral vector, such as a lentiviral vector. In another embodiment, a desirable vector is an adenoviral vector. In still another embodiment, a suitable vector is an adeno-associated viral vector. Adeno, adeno-associated and lentiviruses are generally preferred because they infect actively dividing as well as resting and differentiated cells such as the stem cells, macrophages and neurons. A variety of adenovirus, lentivirus and AAV strains are available from the American Type Culture Collection, Manassas, Va., or available by request from a variety of commercial and institutional sources. Further, the sequences of many such strains are available from a variety of databases including, e.g., PubMed and GenBank.

In one embodiment, a lentiviral vector is used. Among useful vectors are the equine infectious anemia virus and feline as well as bovine immunodeficiency virus, and HIV-based vectors. A variety of useful lentivirus vectors, as well as the methods and manipulations for generating such vectors for use in transducing cells and expressing heterologous genes (RNA or shRNA), e.g., the shRNA that inhibits the expression of Foxp1, are described in N Manjunath et al, 2009 Adv. Drug Deliv. Rev., 61(9): 732-745, incorporated herein by reference. In one embodiment the self-inactivating lentiviral vector (GeMCRIS 0607-793) which was successfully used to transduce T cells directed against tumor cells in leukemia patients (Porter et al., N Engl J Med. 2011 Aug. 25; 365(8):725-33) is useful to carry and express a nucleotide sequence, e.g., that overexpresses or inhibits the expression of Foxp1, as desired herein.

In another embodiment, the vector used herein is an adenovirus vector. Such vectors can be constructed using adenovirus DNA of one or more of any of the known adenovirus serotypes. See, e.g., T. Shenk et al., Adenoviridae: The Viruses and their Replication”, Ch. 67, in FIELD'S VIROLOGY, 6^{th} Ed., edited by B. N Fields et al, (Lippincott Raven Publishers, Philadelphia, 1996), p. 111-2112; U.S. Pat. No. 6,083,716, which describes the genome of two chimpanzee adenoviruses; U.S. Pat. No. 7,247,472; WO 2005/1071093, etc. One of skill in the art can readily construct a suitable adenovirus vector to carry and express a nucleotide sequence as described herein, e.g., an nucleic acid construct that overexpresses Foxp1A/Foxp1D in the cells or an shRNA that inhibits the expression of Foxp1, by resort to well-known publications and patents directed to such viral vectors. See, e.g., Arts, et al, 2003 Adenoviral vectors for expressing siRNAs for discovery and validation of gene function, Genome Research, 13:2325-32.

In another embodiment, the vector used herein is an adeno-associated virus vector. In another embodiment, the vector used herein is an adeno-associated virus (AAV) vector. Such vectors can be constructed using AAV DNA of one or more of the known AAV serotypes. See, e.g., U.S. Pat. No. 7,906,111 (Wilson); Gao et al, Novel Adeno-Associated Viruses From Rhesus Monkeys as Vectors for Human Gene Therapy, PNAS, vol. 99, No. 18, pp. 11854-11859, (Sep. 3, 2002); Rutledge et al, Infectious Clones and Vectors Derived from Adeno-Associated Virus (AAV) Serotypes Other Than AAV Type 2, Journal of Virology, vol. 72, pp. 309-319, (January 1998). One of skill in the art can readily construct a suitable AAV vector to carry and express a nucleotide sequence as described herein by resort to well-known publications and patents directed to such AAV vectors. See, e.g., Grimm et al, Adeno-associated virus vectors for short hairpin RNA expression, Methods Enzymology, 392, 381-405 (2005); U.S. Pat. No. 7,803,611; U.S. Pat. No. 7,696,179.

In yet another embodiment, the vector used herein is a bacterial vector. In one embodiment, the bacterial vector is Listeria monocytogenes. Listeria monocytogenes is a food borne pathogen which has been found to be useful as a vaccine vehicle, especially in attenuated form. See, e.g., Ikonomidis et al, J. Exp. Med, 180:2209-18 (December 1994); Lauer et al, Infect. Immunity, 76(8):3742-53 (August 2008). Listeria monocytogenes are known to spontaneously infect dendritic cells, listerial adhesion factors internalin A and internalin B (Kolb-Mäurer et al, Infection & Immunity, 68(6):3680-8 (June 2000)). Thus, in one embodiment, the bacterial vector is live-attenuated or photochemically inactivated. The heterologous gene of interest, can be expressed recombinantly by the bacteria, e.g., via a plasmid introduced into the bacteria, or integrated into the bacterial genome, i.e., via homologous recombination.

Generally, each of these vectors also comprises a minigene. By “minigene” is meant the combination of a selected nucleotide sequence (e.g., an RNA/DNA sequence that expresses or encodes Foxp1A and/or Foxp1D or a short nucleic acid sequence described herein) and the operably linked regulatory elements necessary to drive translation, transcription and/or expression of the gene product in the host cell in vivo or in vitro. As used herein, “operably linked” sequences include both expression control sequences that are contiguous with the gene of interest and expression control sequences that act in trans or at a distance to control the gene of interest.

These vectors also include conventional control elements that permits transcription, translation and/or expression of the nucleic acid construct in a cell transfected with the plasmid vector or infected with the viral vector. A great number of expression control sequences, including promoters which are native, constitutive, inducible and/or tissue-specific, are known in the art and may be utilized. In one embodiment, the promoter is an RNA polymerase promoter. In another embodiment, the promoter is an RNA polymerase promoter selected from U6, H1, T7, pol I, pol II and pol III promoters. In another embodiment, the promoter is a constitutive promoter. In another embodiment, the promoter is an inducible promoter. In one embodiment, the promoter is selected based on the chosen vector. In another embodiment, when the vector is lentivirus, the promoter is U6, H1, CMV IE gene, EF-1α, ubiquitin C, or phosphoglycerokinase (PGK) promoter. In another embodiment, when the vector is an AAV, the promoter is an RSV, U6, or CMV promoter. In another embodiment, when the vector is an adenovirus, the promoter is RSV, U6, CMV, or H1 promoters. In another embodiment, when the vector is Listeria monocytogenes, the promoter is a hly or actA promoter. Still other conventional expression control sequences include selectable markers or reporter genes, which may include sequences encoding geneticin, hygromicin, ampicillin or purimycin resistance, among others. Other components of the vector may include an origin of replication. Selection of these and other promoters and vector elements are conventional and many such sequences are available [see, e.g., Sambrook et al, and references cited therein].

These vectors are generated using the techniques and sequences provided herein, in conjunction with techniques known to those of skill in the art. Such techniques include conventional cloning techniques of cDNA such as those described in texts [Sambrook et al, Molecular Cloning: A Laboratory Manual, Cold Spring Harbor Press, Cold Spring Harbor, N.Y.], use of overlapping oligonucleotide sequences, polymerase chain reaction, and any suitable method which provides the desired nucleotide sequence.

Thus, in one embodiment, using the information taught herein and publically available and known vector construction components and techniques, one of skill in the art can construct a viral vector (or plasmid) that expresses the desired construct, e.g., a nucleic acid sequence that encodes and thereby can overexpress Foxp1A/Foxp1D or a shRNA that suppresses the expression of Foxp11. In still another embodiment, the vector may be designed to co-express more than one nucleic acid sequence that expresses, overexpresses or inhibits the expression of Foxp1A and/or Foxp1D.

In yet another embodiment, the vector may be designed to co-express a construct that enables targeting of the virus vector to only T cells, T helper cells and/or Tfh cells. Such targeting will enable the virus to be employed in vivo. For example, the virus vector is designed to co-express a T helper cell receptor or a portion of an antibody or fragment to a T helper cell surface marker. Among suitable constructs for co-expression are fragments of monoclonal antibodies targeting T cell surface markers (e.g., CD4). Chimeric receptors may also be co-expressed.

For example, by using the above-noted lentiviral vector (GeMCRIS 0607-793) and transductions at a multiplicity of infection of 5, a high level of expression of chimeric receptors directed against tumor cell antigens can be obtained in >85% primary human T cells (Milone et al., Molecular Therapy (2009) 17 8, 1453-1464). In one embodiment, a minigene or cassette containing a Foxp1A/Foxp1D encoding sequence or shRNA sequence downstream of a RNA polymerase III promoter (e.g., the H1 or the U6 promoters) could be sub cloned into the same lentiviral vector, which would therefore confer expression of the chimeric receptor and expression or silencing of Foxp1A/Foxp1D factor in the same T cell.

In still other embodiments, the viral vectors or plasmids carrying the Foxp1A/Foxp1D nucleic acid construct, e.g., RNA, cDNA or shRNA, are complexed or conjugated to a polymer or any other material that stabilizes the vector or assists in its targeting. Among such stabilizing polymers and materials are polyethyleneimine (PEI), which may be conjugated to the vector, resulting in the generation of nanocomplexes of about 50 nm, as described in Cubillos-Ruiz J R, et al, 2009 J. Clin. Invest., 119(8):2231-44, incorporated by reference herein. In another embodiment, such a stabilizing material is chitosan. In one embodiment, the vector is in a stable composition, with or without conjugation, with cholesterol. In another embodiment, the vector may be conjugated, to an antibody or fragment thereof that permits the vector to be preferentially targeted. In one embodiment, the antibody is an antibody or fragment to a desirable molecule, such as an IL7 receptor. In another embodiment, the antibody is an antibody or fragment to a T cell surface marker, a T cell receptor or a chimeric receptor which also permits targeting. For example, in one another embodiment, the vectors are linked to thiolated F(ab)2 fragments of monoclonal antibodies targeting T helper cell surface markers. In another embodiment, the antibody or fragment is to a T cell receptor or chimeric receptor such as those described above.

C. T Cells for Adoptive Transfer

To generate cells for adoptive transfer, the above-described vectors carrying the minigene expressing at least one Foxp1A/Foxp1D nucleic acid construct (e.g., RNA, DNA or shRNA), and optionally a second construct for co-expression, are delivered to a target T cell. Depending upon the disease for which the method is directed, CD4+ T cells or a subset, such as a Tfh cells may be targeted, which are able to become activated and expand in response to antigen. T cells, useful for adoptive T cell transfer include, in one embodiment, peripheral blood derived T cells genetically modified with suitable receptors. Such receptors are generally composed of extracellular domains comprising a single-chain antibody (scFv) specific for an antigen, linked to intracellular T cell signaling motifs (see, e.g., Westwood, J. A. et al, 2005, Proc. Natl. Acad. Sci., USA, 102(52):19051-19056). In another embodiment, the T cell is a polyclonal or monoclonal T cell, i.e., obtained by apheraesis, expanded ex vivo against antigens presented by autologous or artificial antigen-presenting cells. In another embodiment, the T cell is engineered to express a T cell receptor of human or murine origin.

In certain embodiments, T cells are designed for autologous adoptive transfer into patients. The T cells are engineered ex vivo to express Foxp1A/Foxp1D RNA/DNA or a shRNA capable of down-regulating Foxp1 expression, once the T cells are delivered to the subject. In another embodiment, the subject's T cells can be manipulated in vivo by administration of certain therapeutic agents designed to upregulate or downregulate Foxp1A/Foxp1D activity. Generally, when delivering the vector comprising the minigene by transfection to the T cells, the vector is delivered in an amount from about 5 μg to about 100 μg DNA to about 1×10⁴ cells to about 1×10¹³ cells. In another embodiment, the vector is delivered in an amount from about 10 to about 50 μg DNA to 1×10⁴ cells to about 1×10¹³ cells. In another embodiment, the vector is delivered in an amount from about 5 μg to about 100 μg DNA to about 10⁵ cells. However, the relative amounts of vector DNA to the T cells may be adjusted, taking into consideration such factors as the selected vector, the delivery method and the host cells selected. The vector may be introduced into the T cells by any means known in the art or as disclosed above, including transfection, transformation and infection. The heterologous gene of interest, e.g., the Foxp1A/Foxp1D DNA/RNA or shRNA, may be stably integrated into the genome of the host cell, stably expressed as episomes, or expressed transiently.

In still another embodiment, the T cells are primed/pulsed with and against a selected antigen or otherwise activated before transfection with the vector carrying the Foxp1A/Foxp1D nucleic acid sequence or shRNA. In another example, polyclonal T cells primed against multiple antigens are transduced with the above-described lentiviral vector encoding a Foxp1A/Foxp1D RNA, DNA or shRNA sequence. These adoptive T cells are prepared by pulsing T cells with a selected antigen; transducing the pulsed T cells with a vector expressing a construct that modulates expression of Foxp1A/Foxp1D, and formulating said pulsed, transfected T cells with a suitable pharmaceutical carrier.

The T cells are prepared for adoptive therapy in a suitable pharmaceutical carrier. These T cells are prepared using techniques described in the comparable deletion of CCR5 in T cells administered to HIV infected patients in Perez et al, Nat. Biotechnol. 2008; 26:808-16, which is incorporated by reference herein.

Alternatively, the T cells can be transfected with multiple different viral vectors that express different Foxp1A/Foxp1D RNAs, DNAs or shRNAs, using the same techniques as described above.

D. Small Molecules

In still another embodiment, such a therapeutic agent is a small molecule or drug that up-regulates or down-regulates the expression of Foxp1A and/or Foxp1D and enhances or inhibits the functions or activity thereof.

The compositions comprising the small nucleic acid molecules, viruses, plasmids or T cells described above may be further associated with a pharmaceutically acceptable carrier for in vivo delivery. As used herein the term “pharmaceutically acceptable carrier” or “diluent” is intended to include any and all solvents, dispersion media, coatings, antibacterial and antifungal agents, isotonic and absorption delaying agents, and the like, compatible with administration to humans. In one embodiment, the diluent is saline or buffered saline.

### III. Methods

All of the compositions and components described above may be used in the methods described herein for modulating immune activity.

In one embodiment, a method of modulating the immune response in a mammalian subject comprises modulating the expression or activity of Foxp1 and/or an isoform thereof, in the cells of the subject. In one embodiment, the Foxp1 is the full-length Foxp1A. In another embodiment the Foxp1 isoform is the shorter Foxp1D. In still another embodiment both isoforms 1A and 1D are employed. In one embodiment the targeted cells in which Foxp1A or its isoforms are modulated are CD4+ cells. In another embodiment, the target cells are T follicular helper cells.

As described above, one such method involves increasing or up regulating the expression of Foxp1A, Foxp1D or a combination thereof in the subject's cells in vivo, thereby inhibiting or suppressing B cell response and/or antibody production and/or the activity thereof in the subject. In one embodiment of this method, the B cell response and/or antibody production/activity is reduced or inhibited without depleting the T cell population. The method is particularly useful where the subject has a disease or disorder characterized by excessive B cell response and/or antibody production and/or activity thereof, such as allergy, anaphylaxis, or an autoimmune disorder. According to this embodiment, the method involves delivering to the cells of a subject a nucleic acid construct comprising a sequence encoding Foxp1A, Foxp1D or a combination thereof under the regulatory control of a promoter that expresses or overexpresses the sequence in the cells.

In another embodiment, the method involves decreasing or down regulating the expression of Foxp1A, Foxp1D or a combination thereof in the subject's T cells in vivo, thereby enhancing B cell response and/or antibody production and/or the activity thereof in the subject. In this method, the B cell response and/or antibody production or activity is enhanced without depleting the T cell population. This method is particularly useful in treating subjects having a disease or disorder characterized by insufficient B cell response and/or antibody production or activity, e.g., bacterial infection or cancer. See, e.g., copending U.S. Patent Application No. 61/552,630, incorporated by reference herein. This method can include delivering to the cells of a subject a nucleic acid construct comprising a sequence that reduces or suppresses the expression of Foxp1A, Foxp1D or a combination thereof, e.g., shRNA, siRNA, etc.

These methods can be accomplished using the vectors described above. Alternatively either embodiment of the method can be accomplished by delivering a CD4+ T cell or Tfh cell obtained from the subject, which is transduced or transfected ex vivo with the appropriate nucleic acid construct. As discussed above the T cell is pulsed with a selected antigen, primarily for targeting to T helper or Tfh cells prior to transduction with the nucleic acid construct. Also, the method can include using a virus that permits stable expression of the Foxp1A/Foxp1D construct in the T cell.

In another embodiment, a method of treating a mammalian subject having a disease characterized by excessive B cell response and/or antibody production or activity comprises administering to a subject in need thereof a therapeutic reagent that up-regulates the expression of Foxp1A, Foxp1D or a combination thereof in T cells of the subject.

In still another specific embodiment, a method of treating a mammalian subject having a disease characterized by insufficient B cell production or activity comprises administering to a subject in need thereof a therapeutic reagent that down-regulates the expression of Foxp1A, Foxp1D or a combination thereof in T cells of the subject.

Any of these methods may be accomplished by administering the appropriate construct or composition by any suitable route, including without limitation, intraperitoneal, intravenous, intranasal, or intranodal administration. This administration may be repeated periodically. Alternatively the therapeutic composition is administered ex vivo to a T cell conditioned for adoptive transfer. These methods may further employ administering the nucleic acid construct with a delivery agent, such as a lipid, a cationic lipid, a phospholipid, and a liposome. Further, these methods can comprise administering to the subject another therapeutically active agent useful to treat the disease in question. In certain embodiments, the nucleic acid constructs may be in the form of oligonucleotides or in the form of a nanoparticle complexed with a polymer or other material as described in detail above.

In another embodiment, the method provides administering a vector such as those described in detail above, which specifically infected only T cells, and which contains a construct that expresses, overexpresses, or inhibits the expression of Foxp1A/Foxp1D in a pharmaceutically acceptable carrier or diluent. In one embodiment, where the method the use of a viral vector, target T cells (e.g., helper T cells) are infected by said virus in vivo and Foxp1A/Foxp1D is up-regulated or down regulated in the infected T cells. For this embodiment, the virus specifically infects only T cells. In another embodiment, a plasmid or viral vector comprises the nucleic acid construct, under the control of regulatory sequences. In one embodiment, the viral vector is selected from the group consisting of adenovirus or lentivirus. In another embodiment, the viral vector is complexed with a polymer. In one embodiment, the polymer is PEI, chitosan or any other material that stabilizes the nucleic acid construct. In another embodiment, the method provides administering a viral vector that co-expresses a T helper cell receptor or a chimeric T cell receptor. T cells in the targeted environment become infected by the virus in vivo and Foxp1A/Foxp1D is up regulated or down regulated in the infected T cells.

In another embodiment, the method involves adoptive T cell therapy and involves administering a T cell as described in detail above, e.g., a T cell transduced or transfected ex vivo with the viral vector, wherein the expression of Foxp1 in the T cell is enhanced, extinguished or reduced. As described above, in one embodiment, the viral vector/plasmid is transduced ex vivo into a T cell and said T cell is introduced into the subject. In one embodiment, the T cell is pulsed with a targeting antigen prior to transduction with the viral vector/plasmid. In another embodiment, the T cell has been conditioned for adoptive transfer by pulsing ex vivo with a targeting (antigen-specific) antigen before it is transduced with the virus vector. In still another embodiment, the virus stably expresses the construct in the T cell. Expression of the construct in the T cells transduced ex vivo produces the selected result upon administration to the subject.

The therapeutic compositions administered by these methods, e.g., whether virus, virus nanoparticle, nucleic acid construct alone, nanoparticle, or T cell treated for adoptive therapy,

are administered directly into the subject or into the subject's anatomy most plagued by the disease, where possible. Conventional and pharmaceutically acceptable routes of administration include, but are not limited to, systemic routes, such as intraperitoneal, intravenous, intranasal, intravenous, intramuscular, intratracheal, subcutaneous, and other parenteral routes of administration or intratumoral or intranodal administration. Routes of administration may be combined, if desired. In some embodiments, the administration is repeated periodically.

These therapeutic compositions may be administered to a patient, preferably suspended in a biologically compatible solution or pharmaceutically acceptable delivery vehicle. The various components of the compositions are prepared for administration by being suspended or dissolved in a pharmaceutically or physiologically acceptable carrier such as isotonic saline; isotonic salts solution or other formulations that will be apparent to those skilled in such administration. The appropriate carrier will be evident to those skilled in the art and will depend in large part upon the route of administration. Other aqueous and non-aqueous isotonic sterile injection solutions and aqueous and non-aqueous sterile suspensions known to be pharmaceutically acceptable carriers and well known to those of skill in the art may be employed for this purpose.

The viral vectors or nanoparticles are administered in sufficient amounts to transduce the targeted T cells and to provide sufficient levels of gene transfer and expression to enhance and overexpress or to reduce and inhibit expression of Foxp1A/Foxp1D and provide a therapeutic benefit without undue adverse or with medically acceptable physiological effects, which can be determined by those skilled in the medical arts. The adoptive T cells are similarly administered to express the Foxp1 nucleic acid construct and to increase, reduce or inhibit expression of Foxp1A/Foxp1D to provide a therapeutic benefit without undue adverse or with medically acceptable physiological effects, which can be determined by those skilled in the medical arts.

Dosages of these therapeutic reagents will depend primarily on factors such as the condition being treated, the age, weight and health of the patient, and may thus vary among patients. For example, a therapeutically effective adult human or veterinary dosage of the viral vector or nanoparticle is generally in the range of from about 100 μL to about 100 mL of a carrier containing concentrations of from about 1×10⁶ to about 1×10¹⁵ particles, about 1×10¹¹ to 1×10¹³ particles, or about 1×10⁹ to 1×10¹² particles virus. Methods for determining the timing of frequency (boosters) of administration will include an assessment of disease response to the vector administration. As another example, the number of adoptively transferred T cells can be optimized by one of skill in the art depending upon the response and overall physical health and characteristics of the individual patient. In one embodiment, such a dosage can range from about 10⁵ to about 10¹¹ cells per kilogram of body weight of the subject. In another embodiment, the dosage of T cells is about 1.5×10⁵ cells per kilogram of body weight. In another embodiment, the dosage of T cells is about 1.5×10⁶ cells per kilogram of body weight. In another embodiment, the dosage of T cells is about 1.5×10⁷ cells per kilogram of body weight. In another embodiment, the dosage of T cells is about 1.5×10⁸ cells per kilogram of body weight. In another embodiment, the dosage of T cells is about 1.5×10⁹ cells per kilogram of body weight. In another embodiment, the dosage of T cells is about 1.5×10¹⁰ cells per kilogram of body weight. In another embodiment, the dosage of T cells is about 1.5×10¹¹ cells per kilogram of body weight. Other dosages within these specified amounts are also encompassed by these methods. See, e.g., Dudley et al, 2002, cited above; and Porter et al, 2011, cited above.

In still other embodiments, these methods of down-regulating Foxp1 are part of a combination therapy. In one embodiment, the short nucleic acid molecules, such as siRNA and shRNA, the viral vectors, and the anti-tumor T cells prepared for adoptive immunotherapy as described above, can be administered alone or in combination with various other treatments or therapies for the cancer.

In one embodiment, the methods include IL-7 treatment together with Foxp1A/Foxp1D nucleic acid constructs to the T cell. IL-7Rα is one of the most critical cytokine receptors for T cell survival. The IL-7R complex is composed of IL-7Rα and the common cytokine receptor γ-chain (γ_{c}), but control of IL-7 signaling is primarily dependent on the regulation of IL-7Rα (Mazzucchelli & Durum, 2007, Nat. Rev. Immunol., 7:144-54; Jiang Q et al 2005 Cytokine Growth Factor Rev., 16:513-33). Administration of IL-7 is a synergistic host conditioning strategy together with the adoptive transfer of Foxp1A/Foxp1D nucleic acid construct infected T cells. Exogenous administration of IL-7 is also contemplated.

Thus, in one embodiment, the method further comprises co-administering exogenous IL-7 to the subject. In another embodiment, the therapeutic agent that modulates Foxp1A/Foxp1D expression is provided in combination with a short nucleic acid molecule that targets IL7 Receptor. This molecule can be co-expressed in the vector or in the T cell for adoptive therapy.

In another embodiment, the method further comprises administering to the subject along with the therapeutic agents that either up-regulate or down-regulates Foxp1A/Foxp1D, an adjunctive therapy directed toward the particular disease being treated, which may include a monoclonal antibody, chemotherapy, radiation therapy, a cytokine, or a combination thereof. These therapies may include co-expression of T cell receptor proteins or chimeric T cell receptor proteins in the same virus/plasmids/T cells as described above or administered to the subject in separate viruses/plasmids/T-cells.

In still another embodiment the methods herein may include co-administration or a course of therapy also using other small nucleic acid molecules or small chemical molecules or with treatments or therapeutic agents for the management and treatment of the selected disease. In one embodiment, a method of treatment of the invention comprises the use of one or more drug therapies under conditions suitable for said treatment.

In another embodiment of combination therapy, a passive therapeutic is administered that has immediate effects. In one embodiment, the methods described herein include administration of the Foxp1-modulating therapeutic compositions described above with other known therapies for the selected disease. Additional immune-based or small molecules medicinal therapies can eradicate residual disease. Such combination approaches (i.e., the use of the nucleic acid constructs described and delivered herein, plus other known effective therapies for the disease or its side effects or symptoms) are anticipated to be successful in the treatment of many disease along with the methods described herein.

### III. Examples

The invention is now described with reference to the following examples. These examples are provided for the purpose of illustration only. The compositions, experimental protocols and methods disclosed and/or claimed herein can be made and executed without undue experimentation in light of the present disclosure. The protocols and methods described in the examples are not considered to be limitations on the scope of the claimed invention. Rather this specification should be construed to encompass any and all variations that become evident as a result of the teaching provided herein. One of skill in the art will understand that changes or variations can be made in the disclosed embodiments of the examples, and expected similar results can be obtained. For example, the substitutions of reagents that are chemically or physiologically related for the reagents described herein are anticipated to produce the same or similar results. All such similar substitutes and modifications are apparent to those skilled in the art and fall within the scope of the invention.

### Example 1

### Foxp1 Expression in Activated Cells In Vitro

To determine the role of Foxp1 in T cell immune response, we first examined the expression levels of Foxp1 in activated T cells. Purified CD4⁺ T cells from wild-type C57BL/6 mice were activated by plate-bound α-CD3/α-CD28 antibodies, obtained from ebioscience (anti-CD3; Clone 145.2-C11 and anti-CD28; Clone 37.51) for 2 days. Foxp1 protein expression levels were analyzed in CD4⁺ naïve T cells and in the activated cell using Western blotting with β-actin used as loading control.

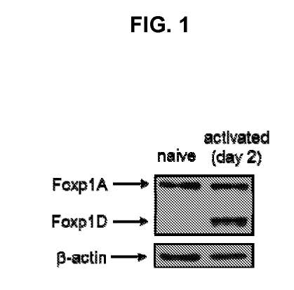

As observed in the Western gel of FIG. 1, while Foxp1A was constitutively expressed in both naïve and activated T cells, the short isoform Foxp1D was mainly induced by T cell receptor (TCR) stimulation.

### Example 2

### Conditional Foxp1A and FOXP1D Transgenic Mice

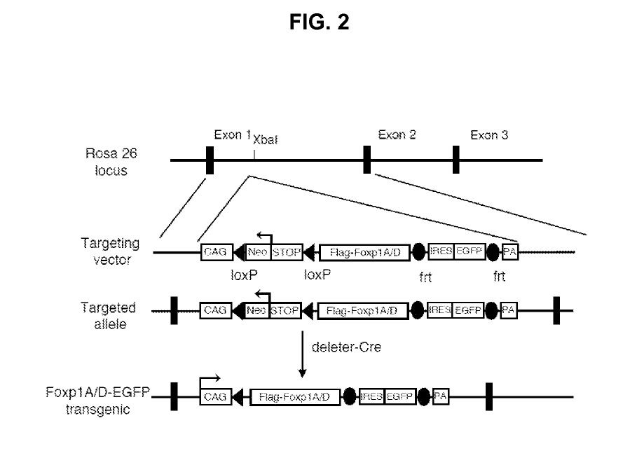

To address the Foxp1 function in T cell immune response, we generated FOXP1A^{Tg}CD4^{Cre} and FOXP1D^{Tg}CD4^{Cre} conditional transgenic mice using the Rosa26-locus knock-in approach (see, e.g., Xiao, C. et al, 2007. MiR-150 controls B cell differentiation by targeting the transcription factor c-Myb. Cell 131:146-159; and the outlined diagram of the transgene of FIG. 2). A key feature of the conditional Rosa26-locus knock-in construct include, as shown in FIG. 2, schematic 1: A cassette with a stop codon flanked by two loxP sites is set in front of the inserted transgene so that the transgene will only be expressed when the stop cassette is deleted by Cre recombinase. Therefore, by using different Cre-deleter mouse strains, the transgene will be expressed in a lineage- and developmental-stage dependent manner. Another key feature is shown in FIG. 2, schematic (2): The inserted transgene is followed by an internal ribosome entry site (IRES) and the sequence encoding the enhanced green fluorescent protein (EGFP); therefore the cells that actively express the transgene will also express the EGFP as a reporter. Still another key feature is shown in FIG. 2, schematic 3: The IRES-EGFP cassette is flanked by frt sites; thus, the EGFP transgene can be deleted with Flp recombinase by crossing the mice with Flp-deleter mouse.

In FOXP1A^{Tg}CD4^{Cre} and FOXP1D^{Tg}CD4^{Cre} mice (all mice are being crossed back to C57BL/6 background), thymocytes at the double-positive (DP) stage start to over-express Foxp1A or Foxp1D transgene.

### Example 3

### Overexpression of FOXP1 in T Cells

We infected FOXP1A^{Tg}CD4^{Cre} or FOXP1D^{Tg}CD4^{Cre} mice as well as wild-type mice (controls which do not overexpress FOXP1) with PR8 flu viruses. At day 10 and day 37 after infection, splenic cells were analyzed.

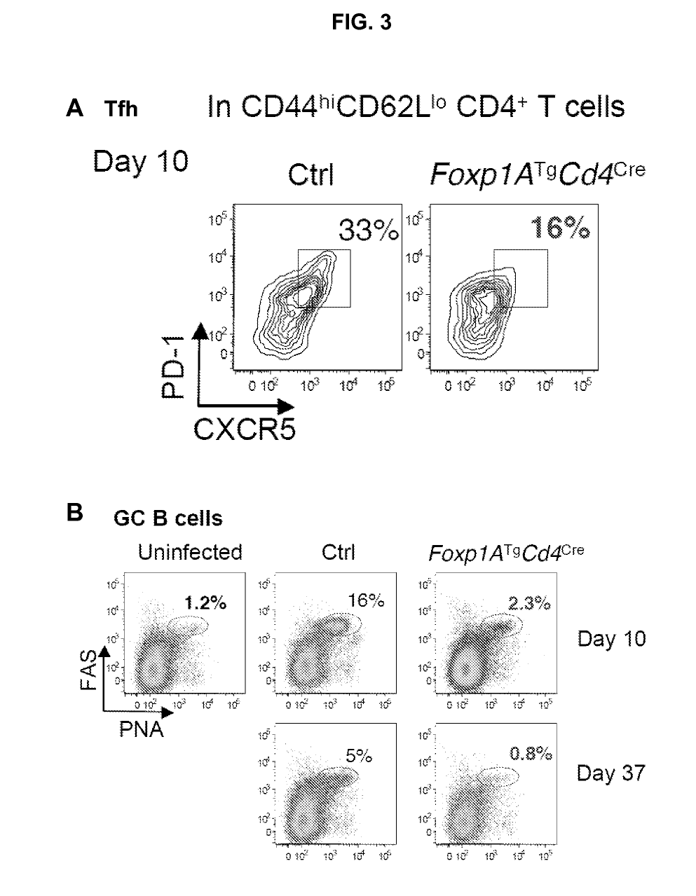

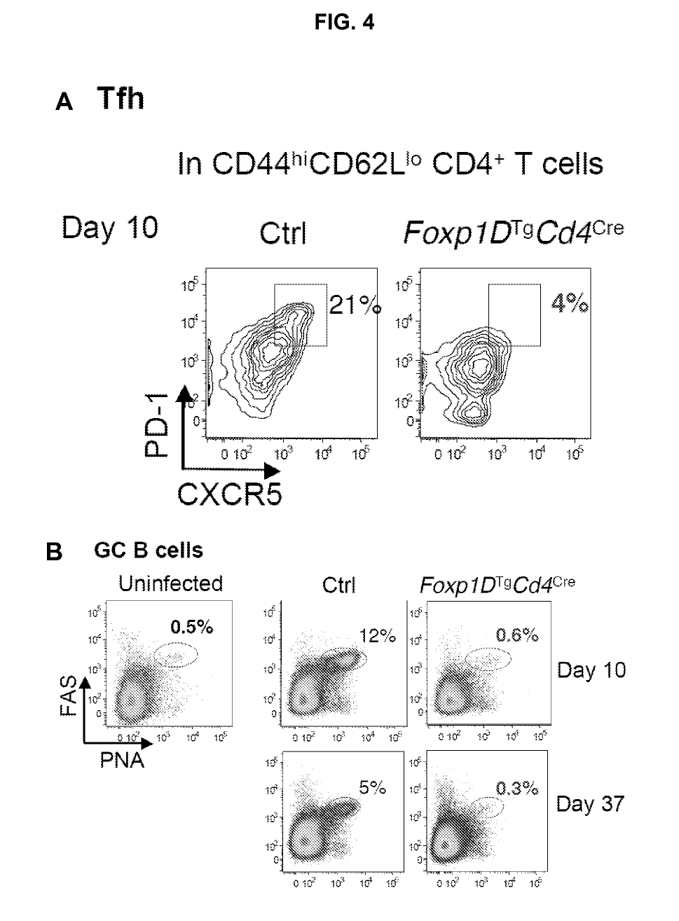

In one analysis, CXCR5⁺PD-1⁺ Tfh cell staining was gated on CD44^{hi}CD62L^{lo} CD4⁺ cells. The results are shown for FOXP1A in the infected control and infected FOXP1A^{Tg}Cd4^{Cre} cells in the histograms of FIG. 3A. The frequencies of CXCR5⁺PD-1⁺ Tfh cells in FOXP1A^{Tg}Cd4^{Cre} mice (16%) were much lower than those in control wild-type mice (33%). The results are shown for Foxp1D in uninfected controls, the infected control and infected FOXP1D^{Tg}Cd4^{Cre} cells in the histograms of FIG. 4A. The frequencies of CXCR5⁺PD-1⁺ Tfh cells in FOXP1D^{Tg}Cd4^{Cre} mice (4%) were much lower than those in control wild-type mice (21%).

In another analysis, germinal center (GC, PNA⁺FAS⁺) B cells were gated on IgD^{low}B220⁺ B cells (i.e., B220 is cell surface marker expressed mostly on B cells). The results are shown for Foxp1A on Day 10 and Day 37 post-infection in uninfected FOXP1A^{Tg}Cd4^{Cre} cells, infected controls (Ctrl), and infected FOXP1A^{Tg}Cd4^{Cre} cells. As demonstrated in FIG. 3B, consistent with lower Tfh cell numbers, we found that the percentages of germinal center (GC) B cells in FOXP1A^{Tg}Cd4^{Cre} mice (2.3%) were dramatically reduced compared to infected controls ((16%) at 10 days; and the percentages of germinal center (GC) B cells in FOXP1A^{Tg}Cd4^{Cre} mice (0.8%) were dramatically reduced compared to infected controls (5%) at 37 days. The uninfected cells showed only 1.2%.

Similar results are shown for Foxp1D on Day 10 and Day 37 post-infection in uninfected FOXP1D^{Tg}Cd4^{Cre} cells, infected controls (Ctrl) and infected FOXP1D^{Tg}Cd4^{Cre} cells. As demonstrated in FIG. 4B, consistent with lower Tfh cell numbers, we found that the percentages of germinal center (GC) B cells in FOXP1D^{Tg}Cd4^{Cre} mice (0.6%) were dramatically reduced compared to infected controls (12%) at 10 days; and the percentages of germinal center (GC) B cells in FOXP1D^{Tg}Cd4^{Cre} mice (0.3%) were dramatically reduced compared to infected controls (5%) at 37 days. The uninfected cells showed only 0.5%.

These data suggest that the subsequent germinal center B cell responses were also suppressed in FOXP1A^{Tg}Cd4^{Cre} or FOXP1D^{Tg}Cd4^{Cre} mice. These data further show that Foxp1A or Foxp1D over-expression in T cells dampens T follicular helper (Tfh) cells as well as B cell responses

These results demonstrate that both Foxp1A and TCR stimulation-induced Foxp1D negatively regulate Tfh cell development as well as the subsequent B cell responses.

### Example 4

### Complementary Deletion Model System

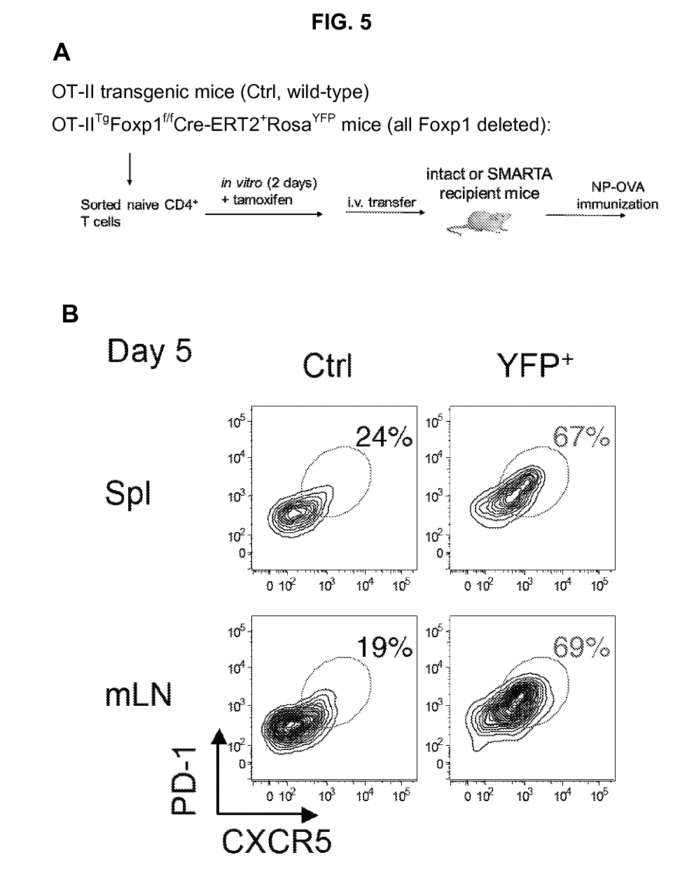

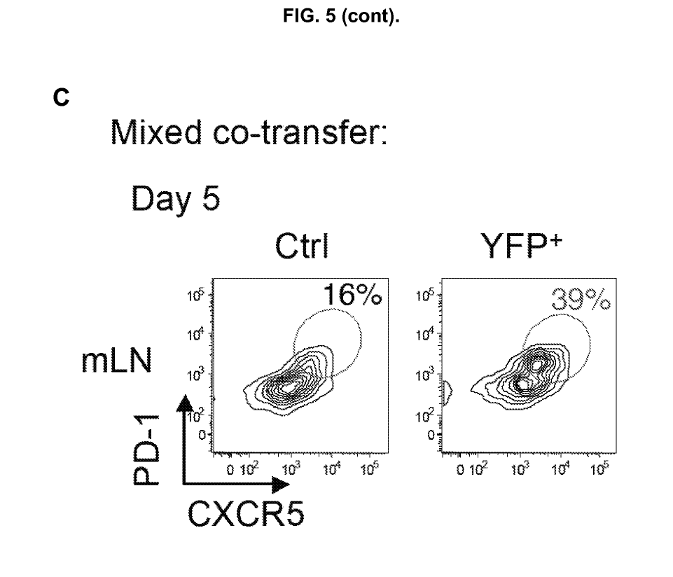

To further confirm that FOXP1 negatively regulates Tfh differentiation, we also set up a complementary deletion model system, in which Foxp1 proteins are inducibly deleted. FIG. 5A is a flow chart diagram of the adoptive transfer. Naïve, purified CD4⁺ T cells were obtained and sorted from wild-type OT-II transgenic (Ctrl) mice or OT-II^{Tg}FOXP1^{f/f}Cre-ERT2⁺Rosa^{YFP} (all Foxp1 deleted) mice. These naïve CD4+ T cells were treated with tamoxifen for two days in vitro. These cells were sorted with wild-type (Ctrl) or yellow fluorescent protein (YFP⁺) cells and transferred into Ly5.1⁺SMARTA TCR transgenic mice or intact Ly5.1⁺C57BL/6 recipient mice. Some recipient mice received both wild-type and Foxp1-deleted OT-II T cells (mixed co-transfer). The recipient mice were immunized with ovalbumin protein conjugated with 4-Hydroxy-3-nitrophenylacetyl hapten (NP-OVA) immediately. Five days after NP-OVA challenge, the splenic cells of the recipient mice were analyzed for CXCR5⁺PD-1⁺ Tfh staining gated on CD44^{hi}CD62L^{lo} CD4⁺ cells.

As shown in the histograms of FIG. 5B, about 20% of wild-type OT-II cells (Ctrl, 24%) in the spleens or the draining lymph nodes (mLN) Ctrl, 19%) of the recipient mice were Tfh cells. In contrast as shown in the histograms of FIG. 5B, the majority (>65%) of Foxp1-deleted OT-II cells in the spleens (YFP+, 67%) or the draining lymph nodes (YFP+, 69%) of the recipient mice differentiated into Tfh cells.

In the recipient mice transferred with both wild-type and Foxp1-deleted OT-II T cells (FIG. 5C), higher percentages of Foxp1-deleted OT-II T cells (39%) differentiated into Tfh cells compared with those of the wild-type OT-II T cells (16%), suggesting a cell-intrinsic control of Foxp1 on Tfh differentiation. These data show that Foxp1 deletion leads to dramatically enhanced Tfh responses.

In summary, the results from the complementary over-expression and deletion experimental model systems clearly demonstrate that Foxp1 exerts a critical negative regulation on Tfh differentiation.

It should be understood that while various embodiments in the specification are presented using “comprising” language, under various circumstances, a related embodiment is also be described using “consisting of” or “consisting essentially of” language. It is to be noted that the term “a” or “an”, refers to one or more, for example, “an anti-tumor T cell” is understood to represent one or more anti-tumor T cells. As such, the terms “a” (or “an”), “one or more”, and “at least one” is used interchangeably herein.

Each and every patent, patent application, including U.S. Provisional Patent Application Nos. 61/637,136, filed Apr. 23, 2012, and 61/636,425, filed Apr. 20, 2012, and publications listed herein, and publically available peptide sequences cited throughout the disclosure, is expressly incorporated herein by reference in its entirety. Embodiments and variations of this invention other than those specifically disclosed above may be devised by others skilled in the art without departing from the true spirit and scope of the invention. The appended claims include such embodiments and equivalent variations.

1. The composition according to claim 32, that up-regulates the expression of Foxp1A, Foxp1D or a combination thereof in T cells for use in the treatment of a disease characterized by excessive B cell response or antibody production or activity in a mammalian subject.

2. A method of modulating the immune response in a mammalian subject comprising modulating the expression or activity of Foxp1, or an isoform thereof or a combination thereof in the cells of the subject.

3. The method according to claim 2, wherein the Foxp1 is the full-length isoform, Foxp1A SEQ ID NO: 1 or isoform Foxp1D SEQ ID NO: 2.

4. (canceled)

5. The method according to claim 2, wherein the cells are CD4+ cells or T follicular helper cells.

6. (canceled)

7. The method according to claim 2, comprising upregulating or increasing the nucleic acid expression or protein expression of Foxp1A, Foxp1D or a combination thereof in the subject's cells in vivo, thereby inhibiting or suppressing B cell response and antibody production or activity in the subject.

8. The method according to claim 2, wherein the B cell response and antibody production is reduced or inhibited without depleting the T cell population.

9. The method according to claim 8, wherein the subject has a disease or disorder characterized by excessive B response or antibody production, wherein the disease is an antibody-mediated disease, or wherein the disease is allergy, anaphylaxis, or an autoimmune disorder.

10-11. (canceled)

12. The method according to claim 7, further comprising delivering to the cells of a subject a nucleic acid construct comprising a sequence encoding Foxp1A, Foxp1D or a combination thereof under the regulatory control of a promoter that overexpresses the sequence in the cells.

13. The method according to claim 2, comprising decreasing or down regulating the nucleic acid expression or protein expression of Foxp1A, Foxp1D or a combination thereof in the subject's T cells in vivo, thereby enhancing B cell response and antibody production in the subject.

14. The method according to claim 13, wherein the B cell response or antibody production is enhanced without depleting the T cell population.

15. The method according to claim 13, wherein the subject has a disease or disorder characterized by insufficient B cell response or antibody production, or wherein the disease is bacterial infection, viral infection or cancer.

16. (canceled)

17. The method according to claim 13, further comprising delivering to the cells of a subject a nucleic acid construct comprising a sequence that reduces or suppresses the expression of Foxp1A, Foxp1D or a combination thereof.

18. The method according to claim 17, wherein the construct comprises a short nucleic acid molecule selected from the group consisting of a short hairpin RNA (shRNA), a short interfering RNA (siRNA), a double stranded RNA (dsRNA), a micro RNA, and an interfering DNA (DNAi) molecule, optionally under the control of a suitable regulatory sequence.

19. The method according to claim 12, wherein the nucleic acid construct is a plasmid or viral vector, wherein the vector is a non-pathogenic virus, or wherein the vector is a viral vector selected from the group of lentiviral, adenoviral or retroviral vectors.

20-21. (canceled)

22. The method according to claim 2, further comprising delivering a CD4+ T cell obtained from the subject, which is transduced or transfected ex vivo with the nucleic acid construct, or wherein the T cell is pulsed with activation prior to transduction with the nucleic acid construct, or wherein the virus stably expresses the construct in the T cell.

23-24. (canceled)

25. A method of treating a mammalian subject having a disease characterized by abnormal B cell response or antibody production or activity comprising:
(a) administering to a subject in need thereof a therapeutic reagent that up-regulates the expression of Foxp1A, Foxp1D or a combination thereof in T cells of the subject, when the subject's disease is characterized by excessive B cell response or antibody production or activity; or
(b) administering to a subject in need thereof a therapeutic reagent that down-regulates the expression of Foxp1A, Foxp1D or a combination thereof in T cells of the subject, when the subject's disease is characterized by insufficient B cell response or antibody production or activity.

26. (canceled)

27. The method according to claim 25, comprising:
administering the composition by intraperitoneal, intravenous, intranasal, or intranodal administration; or
administering the composition periodically; or
administering said agent ex vivo to a T cell conditioned for adoptive transfer; or
administering the composition or agent with a delivery agent selected from the group consisting of a lipid, a cationic lipid, a phospholipid, and a liposome; or
administering to the subject another therapeutically active agent useful to treat the disease.

28-31. (canceled)

32. A therapeutic or prophylactic composition comprising a nucleic acid construct or small molecule that modulates the expression of Foxp1A, Foxp1D, or a combination thereof, and a pharmaceutically acceptable carrier or diluent.

33. The composition according to claim 32, wherein the carrier or diluent is saline or buffered saline.

34. The composition according to claim 32, that down-regulates the expression of Foxp1A, Foxp1D or a combination thereof in T cells for use in the treatment of a disease characterized by insufficient B cell response or antibody production or activity in a mammalian subject.


SEQUENCE LISTING (6 sequences)

SEQ ID NO: 1 - DNA, 7102 bp, Homo sapiens
  CDS (527)..(2560)
ggggggtgggcgccagcgccccggcgaacggcaaagagggagccgctcccgctcggggggccgctggagtgcccagcgggaacccgaaagtttgtaagaggaagagagcgcgcggcgagcgagcgagcgggccgggggcagcggcagcggcgccggggaccatggtgctgccggcgcctcctccgcgggcgtgaaggcggcgctcctactccctccccggactccgcggtgtcccagaagcttttgttgacaattccagtttccgaacaaaacatttcggcaatggtgagggcttcgatcccttctctgatttgctgtcagccatgaacggatggatgtgatgcctgctagccaaaaggcttccctctgtgtgttgcagtcctgtggcattatgcatgccccctcccagtgaccccaggctttttatggctgtgagacacgttaaaatttcaggggtaagacgtgaccttttgaggtgactataactgaagattgctttacagaagccaaaaaaggtttttgagtcatgatgcaagaatctgggactgagacaaaaagtaacggttcagccatccagaatgggtcgggcggcagcaaccacttactagagtgcggcggtcttcgggaggggcggtccaacggagagacgccggccgtggacatcggggcagctgacctcgcccacgcccagcagcagcagcaacaggcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtaaaaaaaaaa

SEQ ID NO: 2 - protein, 677 aa, Homo sapiens
MMQESGTETKSNGSAIQNGSGGSNHLLECGGLREGRSNGETPAVDIGAADLAHAQQQQQQALQVARQLLLQQQQQQQVSGLKSPKRNDKQPALQVPVSVAMMTPQVITPQQMQQILQQQVLSPQQLQVLLQQQQALMLQQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTGNNHSSLDLTTTCVSSSAPSKTSLIMNPHASTNGQLSVHTPKRESLSHEEHPHSHPLYGHGVCKWPGCEAVCEDFQSFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGNLASAIREELNGAMEHTNSNESDSSPGRSPMQAVHPVHVKEEPLDPEEAEGPLSLVTTANHSPDFDHDRDYEDEPVNEDME

SEQ ID NO: 3 - DNA, 6394 bp, Homo sapiens
  CDS (119)..(1852)
cagtccgcctgcacgccgaggccgccggaccccacgcccggcccgcgcgggcgccccgagcccggctccgcgcgcctggcggctacatggagagtgtcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtaaaaaaaaaa

SEQ ID NO: 4 - protein, 577 aa, Homo sapiens
MMTPQVITPQQMQQILQQQVLSPQQLQVLLQQQQALMLQQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTGNNHSSLDLTTTCVSSSAPSKTSLIMNPHASTNGQLSVHTPKRESLSHEEHPHSHPLYGHGVCKWPGCEAVCEDFQSFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGNLASAIREELNGAMEHTNSNESDSSPGRSPMQAVHPVHVKEEPLDPEEAEGPLSLVTTANHSPDFDHDRDYEDEPVNEDME

SEQ ID NO: 5 - RNA, 19 nt, Homo sapiens
aaucugggacugagacaaa

SEQ ID NO: 6 - RNA, 19 nt, Homo sapiens
gaugcaagaaucugggacu